(19) United States
(12) Patent Application Publication
(10) Pub. No.: US 2010/0311111 A1
(43) Pub. Date: Dec. 9, 2010

(54) Methods for Optimizing Production and Collection of Reusable Gases

(76) Inventors: Rex S. Spendlove, Millville, UT (US); Randall Thunell, River Heights, UT (US); Gary Burningham, North Logan, UT (US); Salam Awada, Logan, UT (US); Paul Manning, North Logan, UT (US)

Correspondence Address: KEELY SCHNEITER, 399 NORTH MAIN, SUITE 300, LOGAN, UT, 84321, US

(21) Appl. No.: 12/790,559

(22) Filed: May 28, 2010

Related U.S. Application Data
(60) Provisional application No. 61/181,759, filed on May 28, 2009.

(51) Int. Cl. C12P 5/02; C12N 1/20; B01D 53/00; C01B 31/20

(52) U.S. Cl. 435/42; 435/167; 435/252.4; 423/437.1; 422/285

(57) ABSTRACT

Methods for increasing methane emitted from organic waste and for collecting methane and other gaseous bi-products. A gas collection unit collects gas in a first separation tank, wherein the gas is separated into methane and other components. The methane is collected, and other components are diverted. A second separation tank receives diverted components combined with an acidic solution and further separates the components into carbon dioxide and other compounds. The carbon dioxide is collected for reuse in a variety of applications.

### CROSS-REFERENCE TO RELATED APPLICATIONS

This application claims priority to U.S. Provisional Patent Application No. 61/181,759, filed May 28, 2009, which is incorporated herein by reference in its entirety.

### BACKGROUND OF THE INVENTION

1. The Field of the Invention

This invention relates generally to virus resistant microorganisms and methods to separate gases. More particularly, embodiments of the present invention relate to processes for using virus resistant microorganisms in applications relating to the aerobic digestion of waste and the separation of gases produced during such processes.

2. Related Technology

Methane is one of the primary contributors to global warming. Though methane is mentioned less frequently than carbon dioxide as a gas that contributes to global warming, a molecule of methane absorbs twenty to twenty-six times as much heat as a molecule of carbon dioxide.

Methane is released from a variety of different sources, such as, for example, sewage lagoons, ponds, garbage landfills, coal-fired plants, uncapped natural gas wells on land and in the ocean, swampy areas, and from certain ponds and lakes that contain organic matter.

While some methane is produced from these sources spontaneously, other methane emissions result as a bi-product from biological and/or chemical processes, or may be increased from these sources by such processes. For example, manure that is biologically treated may release more methane than untreated manure.

As manure and other organic matter is treated, bacteria are often used to aid in digestion of the manure. Bacteria and other microorganisms, however, are susceptible to infection by viruses. Certain estimations place the number of viruses in the environment at an amount ten times that of the number of bacteria. Many of the bacteria involved in the digestion of manure are, therefore, killed by viruses. The population of bacteria present to digest manure can be restored by the growth of bacteria that mutate and become resistant to infection, thus creating a great fluctuation in the bacterial population levels involved in the digestion of manure. Such fluctuations in the bacterial population levels affect the amount of methane produced by processes using bacteria to digest manure or other matter. Moreover, methane released from areas where such processes take place is often released directly into the air.

Embodiments of the present invention provide ways to increase methane produced in anaerobic digestion of manure and other organic matter and collect methane produced in these processes, thereby maximizing methane produced in waste management processes, preventing methane from being released directly into the air, and collecting methane to be used for a variety of energy efficient purposes. Embodiments of the present invention may also be used to collect gases released from coal-fired plants and other sources of gaseous waste and to separate and collect these gases for use in a variety of applications.

### BRIEF SUMMARY OF THE INVENTION

Embodiments of the present invention include methods for increasing production of methane from human and animal waste. The methane and other gases produced are collected and separated in gas collection units. These gases are then collected for use in other applications, rather than being emitted into the air as waste.

These and other aspects of the present invention will become more fully apparent from the following description and appended claims, or may be learned by the practice of the invention as set forth hereinafter.

### BRIEF DESCRIPTION OF THE DRAWINGS

To further clarify the above and other aspects of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The drawings are not drawn to scale. The invention will be described and explained with additional specificity and detail through the use of the accompanying drawings in which:

FIG. 1 shows a chart depicting a process for increasing methane production from waste products;

FIG. 2 shows an embodiment of a gas separation container; and

FIG. 3 shows a chart depicting a process for determining phage resistance of bacteria.

### DETAILED DESCRIPTION OF EXEMPLARY EMBODIMENTS

Embodiments of the present invention relate to increasing production of methane from organic waste and collection and separation of methane and other gases. Methane and other gases are emitted from a variety of sources, such as, for example, coal-fired plants, outdoor sewage lagoons, and so forth. For sources of methane produced due to anaerobic digestion of organic material, embodiments of the present invention provide methods for increasing methane production, while decreasing production of other undesirable gases. In addition, embodiments of the present invention provide a gas collection unit for collecting methane (CH₄) from a variety of sources, include bio-gas and hot smoke stack gas sources.

Embodiments of the present invention show anaerobic system using bacteria resistant to bacteria viruses, often referred to as bacteriophages or phages, to produce methane from manure. These bacteria that are resistance to bacteria viruses are also referred to as phage-resistant bacteria. While descriptions are provided for preparing phage resistant bacteria, embodiments of the present invention also apply to other microorganisms such as, for example, protozoa and fungi. As shown in embodiments of the present invention, these virus resistant microorganisms can be used is the aerobic and anaerobic treatment of all human and animal waste. Moreover, as shown in other embodiments of the present invention, virus resistant microorganisms can also be used in the production of methane from cellulose and sugars.

The digestion of cellulose to produce methane begins in the rumen, a part of the stomach, of animals. In the rumen, a steady state exists between bacteria and viruses that attack these bacteria. Within the rumen, bacteria susceptible to infecting viruses may be partially or totally destroyed by viruses, depending on the susceptibility of the bacteria to the viruses. The destruction of bacteria by viruses may also depend on the burst size of the viruses.

As virus-sensitive bacteria are destroyed, other bacteria that are of the same species as the destroyed bacteria but that are resistant to the infecting virus or viruses, grow to keep the population of bacteria and viruses in balance. Over time the population of bacteria develops into a more resistant population. While this new population may be more resistant to viruses than the original population, the new population may still be susceptible to new viruses that may appear, or to viruses that may adapt. This new population of bacteria, also referred to as phage-resistant bacteria, is parts of a mixed group of bacteria and other microorganisms found in the rumen.

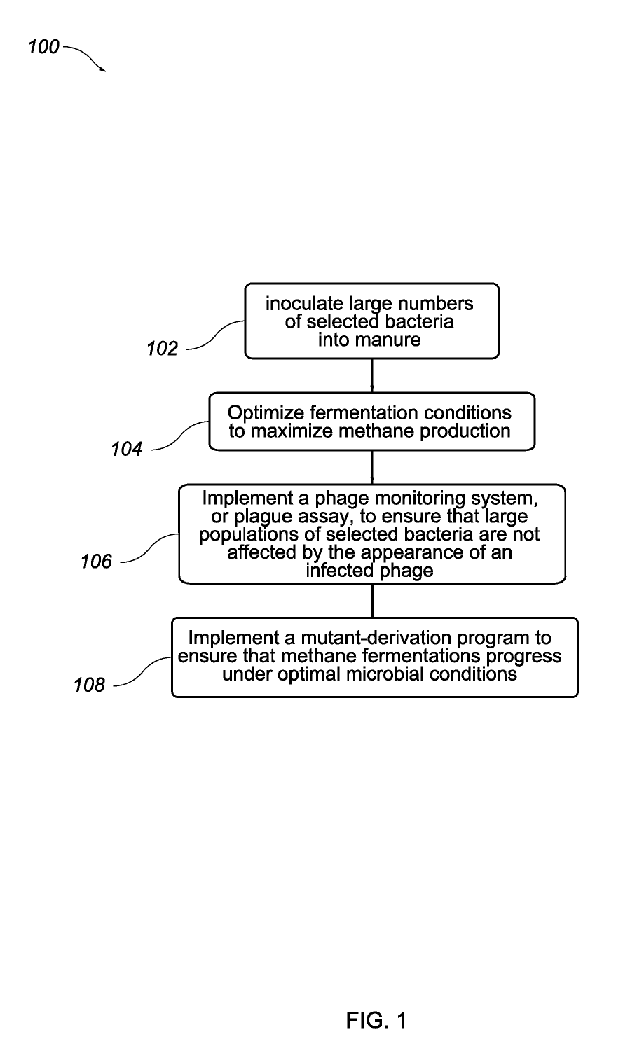

While this group of microorganisms is most beneficial for the health of the animal, it may not represent the best microbial population for the production of methane. Thus, as shown in stage 102 of FIG. 1, embodiments of the present invention include inoculating large numbers of selected bacteria into animal manure. The selected bacteria refer to bacteria that represent the best microbial population for the production of methane.

After the selected bacteria are inoculated into the manure, embodiments of the present invention provide for optimization of fermentation conditions to maximize the production of methane, as shown at stage 104. In one embodiment of the invention, optimization of fermentation conditions includes, for example, optimizing the pH, temperature, substrate, moisture conditions, nutrients, and so forth. In one embodiment of the invention, as shown at stage 106, a phage monitoring system, or plague assay, is implemented to make certain that the large populations of selected bacteria are not affected by the appearance of an infected phage.

In addition to implementation of a phage monitoring system, a mutant-derivation program is also implemented. The mutant-derivation program ensures that methane fermentations progress under optimal microbial conditions. This is shown in FIG. 1 at stage 108.

With attention now to FIG. 1, chart 100 shows a method for increasing methane production from organic solids. At stage 102, large numbers of selected bacteria are inoculated into organic matter to “out compete” other organisms. At stage 104, fermentation conditions are optimized to maximize the production of methane. Such optimization may include, for example, optimization of pH, temperature, substrate, and so forth. Then at stage 106, a phage monitoring system, and mutant-derivation program are implemented to ensure that methane fermentations are progressing under optimal microbial conditions. Moreover, the presence of undesirable gases in the system is reduced, as shown at stages 108 and 110. First, at stage 108, phages are added to anaerobic digesters to kill microorganisms that digest proteins to produce undesirable gases. Examples of undesirable gases include hydrogen sulfide, nitrogen oxides, and ammonia. Finally, at stage 110 organic waste is inoculated with large numbers of cellulose digesting bacteria so that protein digesting bacteria is unable to compete for nutrients. Thus, the growth of protein digesting bacteria is greatly restricted and the emission of undesirable gases is also restricted.

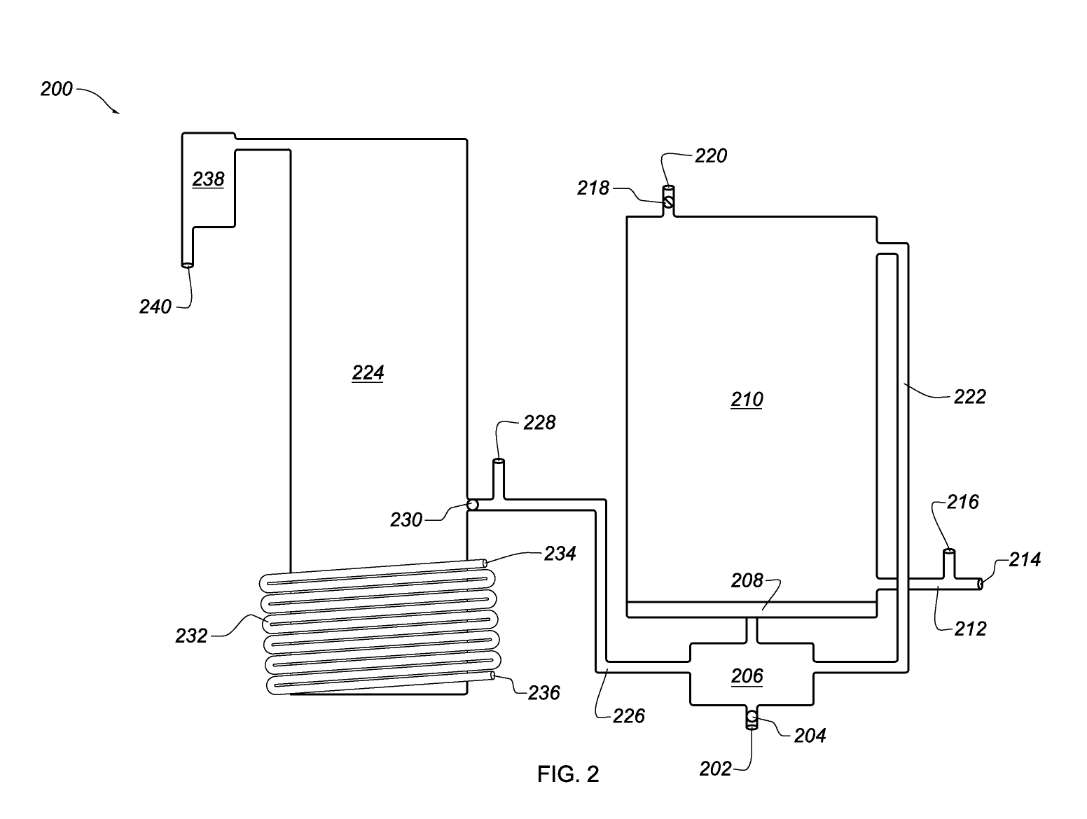

With attention now to FIG. 2, a gas collection and separation unit 200 is shown. As noted above, gas collection unit 200 may be used in such applications as, for example, collection of bio-gas and collection of hot smoke stack gas, such as hot smoke stack gas released from coal-fired plants. Gas collection units of the present invention may be constructed of plastics, such as, for example, soft, gas-impermeable, inflatable plastic, or hard plastic. Gas collection unit 200 includes opening 202, entry pump 204, heat exchanger 206, and sparger 208. Gas collection unit 200 also includes a first separation tank 210, and pipe 212, having openings 214 and 216. Further, a valve 218 and emission opening 220, and a pipe 222, connect the top of first separation tank 210. Pipe 222 also connects to heat exchanger 206.

Gas collection unit 200 also includes a second separation tank 224, connected to heat exchanger 206 by pipe 226. Pipe 226 further includes influx opening 228 and a pressure release valve 230. Cooling coils 232, located at the bottom of second separation tank 224, include input opening 234 and output opening 236. Compressor 238, attached to the top of second separation tank 224, includes opening 240.

In operation, bio-gas or hot smoke stack gas enters opening 202 and moves through entry pump 204 to heat exchanger 206. In applications where bio-gas is collected in gas collection unit 200, the bio-gas is first assayed to determine the ratio of the volume of methane to the volume of carbon dioxide. This ratio is then used to determine the location of the entry port of the bio-gas into the gas collection unit, or, stated differently, to determine where the entry port should be placed. In one embodiment of the invention the entry port is opening 202.

From heat exchanger 206, the gas moves into, or may be, in other instances, pumped into, sparger 208. Sparger 208 introduces the gas into water, such as alkaline water, contained in first separation tank 210. Water enters first separation tank 210 through opening 214 attached to pipe 212, while sodium hydroxide (NaOH) enters pipe 212 through opening 216. The water present in first separation tank 210 increases the solubility of carbon dioxide (CO₂) gas entering into first separation tank 210. Hydroxide, carbonates, and bicarbonate salts of sodium, potassium, ammonium, and so forth, can be used to produce alkalinity solutions. Alkaline water saturated with carbon dioxide will remain in solution during the release of methane.

As methane is released, the methane is exhausted through valve 218 and emission opening 220. Water soluble gases, such as carbon dioxide and hydrogen sulphide (H₂S) circulate through pipe 222 and pass through heat exchanger 206, once the solution in first separation tank 210 is saturated.

As the gas solution passes through pipe 226, acidic solution is added to the gas solution phase through influx opening 228. Pressure release valve 230 releases the acidic solution, as a mist, into second separation tank 224. The heat, acidic solution, and misting combine to help increase the rate at which carbon dioxide gas is released in second separation tank 224.

Cooling coils 232, having an opening 234 for water input and an opening 236 for water output, help with water condensation and gas separation. During the summer and warmer months, the coils may be cooled by a refrigeration unit. During winter and colder months, the coils are cooled by the ambient air. Alternatively, moisture can be removed by passing gases through a desiccant before or after the gases pass through second separation tank 224.

As carbon dioxide is collected in second separation tank 224, it passes into compressor 238 and is collected through opening 240. After collection, the carbon dioxide can be used in a variety of different applications, including, for example, oil sequestration in oil fields.

With continued attention to FIG. 2, the following description also shows functionality of the gas collection and separation unit 200. In one embodiment of the invention, unit 200 is configured to be attached to or coupled to a covering placed over a methane production source. For example, unit 200 may be attached to a covering that is placed over a methane source, such as, for example, organic solids. In one embodiment of the invention, unit 200 may be attached to a covering that covers organic solids whose methane production has been enhanced by processes described in reference to FIG. 1. In other embodiments of the invention, unit 200 may be couple to hosing extending from a covering of a methane source, rather than attaching directly to the covering. In yet another embodiment of the invention, unit 200 may attach to or be couple to other methane sources, such as, for example, smoke stacks from coal-fired plants, natural gas wells, and any other methane source.

Methane released from a source enters unit 200 through opening 202. In one embodiment of the invention, methane is further drawn into unit 200 by entry pump 204 and pumped through sparger 208. In one embodiment of the invention, sparger 208 is a unit configured with fine or very fine holes through which the gas passes, thus increasing the surface area of the gas. Passing the gas through a sparger in this way may increase the solubility of the gas. For example, in one embodiment of the invention where carbon dioxide enter unit 200 through opening 202 and is pumped by entry pump 204 through sparger 208, the rate of carbon dioxide solubility is faster because the surface area of carbon dioxide bubbles that are created to pass through water contained in first separation tank 210 is increased. This effect, increasing the solubility of carbon dioxide by breaking up large carbon dioxide bubbles into small carbon dioxide bubbles, is achieved by passing the carbon dioxide through a sparger, such as, for example, sparger 208.

As noted above, after passing through sparger 208, gas enters alkaline water tank 210. Alkaline water tank 210 contains water with increased alkalinity. The increased alkalinity of water tank 210 functions to increase the solubility of carbon dioxide in the tank 210. In one embodiment of the invention, hydroxide, carbonates, bicarbonates of salts and sodium, potassium, ammonium, and other substances may be used to create the alkaline solution. While methane will be released from the alkaline water solution held in tank 210, the alkaline water saturated with carbon dioxide will remain in solution during the release of the methane. In one embodiment of the invention, methane is exhausted through opening 220. In one embodiment of the invention, opening 220 connects to an exhaust valve 218.

While methane from bio-gas or smoke-stack gas that has entered the unit 200 through opening 202 may be released through opening 220, water soluble gases, including, for example, carbon dioxide and hydrogen sulfide, circulate through passage 222 after the water in tank 210 becomes saturated. In one embodiment of the invention, such water soluble gases pass through heat exchange 206 and into passage 226. These gases enter tank 224. In one embodiment of the invention, an acidic solution is added to the gas solution phase of the gases that are entering tank 224. In another embodiment of the invention, the acidic solution is released into tank 224 through a pressure release valve 230. Thus, in one embodiment of the invention, the acidic solution enters tank 224 as a mist. The heat contributed by heat exchange 206, the addition of the acidic solution, and the misting help the carbon dioxide gas to be released more quickly in the tank 224. The process of gas separation is further enhanced by cooling coils 232 that function in part to condense water, such as, for example, water vapor that may be saturated with carbon dioxide. Finally, compressor 238 compresses the carbon dioxide with passes through opening 240 and may be collected for a variety of uses, including, for example, oil sequestration in oil fields. Embodiments of the gas collection and separation unit as described with reference to FIG. 2 may be used in conjunction with anaerobic systems used in the production of methane as described herein and with systems and methods as described with reference to FIGS. 1, 3, and 4.

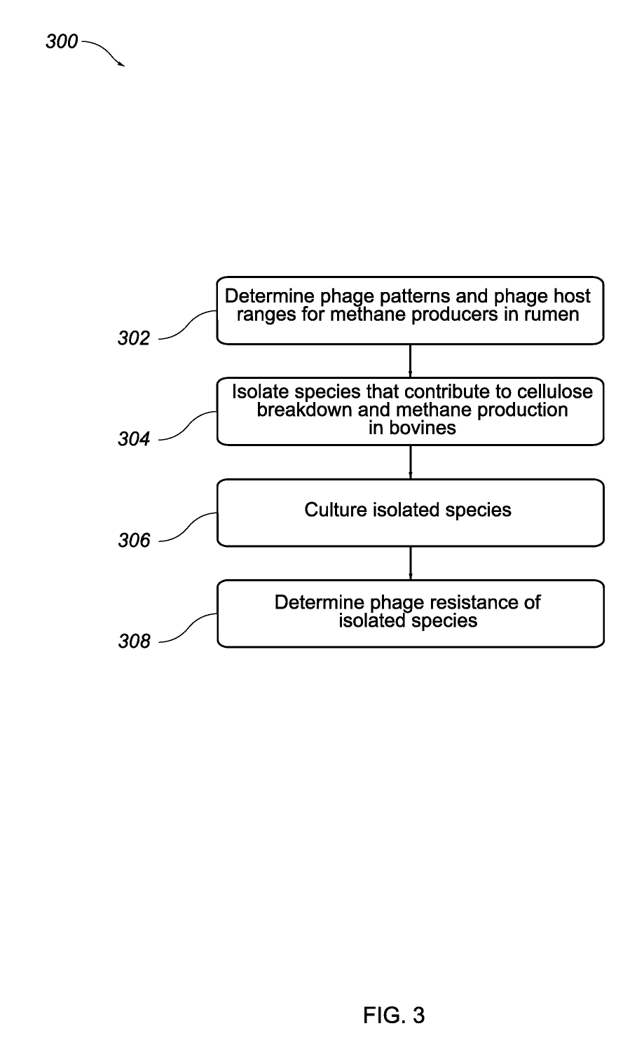

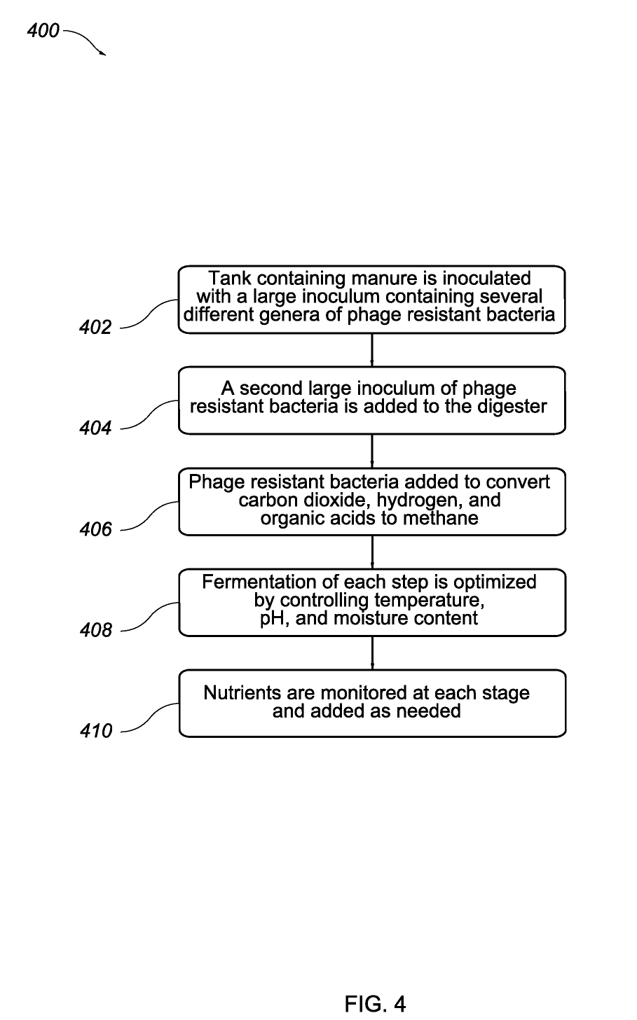

With attention now to FIG. 3, a process for determining phage patterns and phage host ranges for bacteria is shown. First, at stage 302, phage patterns and phage host ranges for methane producers in rumen are determined. The species that contribute to cellulose breakdown and methane production in bovines are determined, as shown at stage 304. The isolated species are cultured, as shown at stage 306, and the phage resistance of the isolated species is determined, as shown at stage 308.

Within the rumen of bovines, one of the primary cellulose digesting bacteria is Streptococcus bovis, which exhibits a greatly reduced phage host range. Such bacteria are typically infected only by a single or very small number of phages. Embodiments of the present invention include methods for determining the phage patterns and phage host ranges for Methanobacter, the primary producer in the rumen of bovines. Moreover, embodiments of the present invention further include the isolation and culture of other species that work synergistically with the Methanobacter and Streptococci, in addition to a determination of their phage resistance. Finally, additional embodiments of the present invention include the addition of phage resistant cellulose digesting bacteria to the feed of animals to increase the rate of cellulose digestion, thus increasing milk production, and the addition of Escherichia coli phages to animal feed in feed lots prior to slaughter to reduce E. coli contamination in meat.

With attention now to FIG. 4, a process for anaerobic digestion of manure by phage resistant bacteria is shown. In one embodiment of the invention, the anaerobic digestion of manure by phage resistant bacteria may take place in tanks containing a mixing device. A tank containing manure is inoculated with a large inoculum containing several different genera of phage resistant bacteria that digest cellulose, as shown at stage 402. These bacteria outgrow the bacteria originally present in the manure. As the cellulose is digested the end product is organic solids. When the cellulose is mostly digested to form organic acids, a second large inoculums of phage resistant bacteria is added to the digester, as shown at stage 404. These bacteria also outgrow microorganisms in the digester and convert the organic acids products in the first fermentation to carbon dioxide, hydrogen, and some small organic salts.

As shown at stage 406, the phage resistant bacteria added at the third stage of the digestion convert the carbon dioxide, hydrogen, and organic acids into methane. Throughout the digestion, the pH, moisture content, and temperature, are monitored and adjusted as needed to optimize each fermentation step, as shown at stage 408. Nutrients are also monitored during each stage of fermentation and added as needed, as shown at stage 410. In one embodiment of the invention, protozoa and fungi, which participate synergistically with bacteria in the production of methane from manure, are also used in the process described above. In one embodiment of the invention, these techniques are applied to the aerobic digestion of human sewage and garbage.

Moreover, techniques included in embodiments of the present invention may be adapted for aerobic digestion of manure and waste such as that present in windrows, and any other aerobic system. Embodiments of the present invention further include using phage resistant bacteria to digest or pre-digest animal feed, grain, corn silage, and/or straw to improve feed efficiency and meat and milk production. Further, phage resistant bacteria may be used, in embodiments of the present invention, to treat straw, thatch, and crop residues in agricultural fields and post-harvest and pre-planting to breakdown residues and enhance the production of organic matter. In one embodiment of the invention, the inoculums is sprayed directly on crop residues and/or the soil and incorporated into the soil. Finally, embodiments of the present invention may further be used for thatch breakdown in instances such as after lawns are mowed on golf courses. In such instances phage resistant bacteria may be used to breakdown residues.

Thus, embodiments of the present invention provide methods for not only anaerobic digestion of waste and increasing methane production from a variety of different waste sources, but also collecting and separating gases, such as, for example, bio-gases and hot smoke stack gases released from such sources as coal fired plants. Gases collected and separated according to embodiments of the present invention can then be used in a variety of different ways, rather than being discharged as waste into the atmosphere.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A gas collection and separation unit, comprising:
an intake opening connected to a first tank;
a first tank containing water and having an emission opening for the release of methane;
a sparger located within the first tank;
a passageway connected to the first tank for the circulation of water soluble gases, the passageway further connecting to a heat exchanger;
a conduit for water soluble gases attached to the heat exchanger;
an opening connected to the conduit for introduction of acidic solution into the water soluble gases;
a pressure release valve attached to an end of the conduit for release of the acidic solution and the water soluble gases into a second tank, wherein the second tank includes a condenser; and
a compressor for compressing carbon dioxide, the compressor further including an opening for emitting carbon dioxide.

2. The unit as recited in claim 1, further comprising:
an entry pump connected to the input opening.

3. The unit as recited in claim 1, further comprising:
a water connection pipe connected to the first tank such that water enters the tank from the water opening.

4. The unit as recited in claim 3, wherein an input line further connects to the water connection pipe to allow introduction of substances into the water connection pipe to create alkalinity solutions.

5. The unit as recited in claim 1, wherein the first tank includes an exhaust valve for regulating the release of methane from the emission opening.

6. The unit as recited in claim 1, wherein the condenser further includes a first opening for water input and a second opening for water output.

7. A gas collection and separation unit, comprising:
a heat exchanger, wherein gas enters the receptacle near the heat exchanger, connected to a sparger, wherein the sparger is further connected to a first separation tank;
the first separation tank further including a valve and a discharge pipe, wherein the valve is configured to release methane collected within the first separation tank, and the discharge pipe is configured to release water-soluble gases after saturation, and wherein the discharge pipe is further connected to the heat exchanger;
a connector pipe connecting the heat exchanger to a second separation tank, wherein the water-soluble gases of the discharge pipe are combined with an acidic solution and passed through a pressure release valve to enter the second separation tank as a mist;
cooling coils connected to the bottom of the second separation tank, having an input opening and an output opening; and
a compressor connected to the top of the second separation tank wherein carbon dioxide collected in the second separation tank moves through the compressor to a collection opening.

8. A method for collecting and separating gases of different densities, comprising:
receiving gaseous emissions into an opening connected to a heat exchanger;
combining the gaseous emission with an alkaline water;
filtering the gaseous emissions through a sparger;
collecting the gaseous emission in a first separation tank;
capturing methane separated from the gaseous emissions;
diverting water soluble gases used in the first separation tank to the heat exchanger;
combining the water soluble gases with an acidic solution;
releasing the water soluble gases and acidic solution as a mist into a second separation tank;
collecting water droplets from the second separation tank in cooling coils; and
capturing carbon dioxide separated in the second separation tank and collected in a compressor.

9. The method as recited in claim 8, further comprising:
receiving water and one or more of hydroxide, carbonates, bicarbonates of salts of sodium, potassium and ammonium for creating an alkaline water solution.

10. The method as recited in claim 8, further comprising:
cooling gases received in the second separation tank.

11. The method as recited in claim 8, further comprising:
pumping gasesous emissions into the first separation tank through the sparger.

12. A method for production of methane, comprising:
inoculating organic waste with large numbers of selected bacteria;
optimizing fermentation conditions to maximize methane production;
implementing a phage monitoring system and a mutant-derivation program to ensure optimal microbial conditions in organic matter degeneration.

13. A method for digestion of organic matter, comprising:
determining phage patterns and phage host ranges of methane producers in rumen;
isolating species of bacteria that contribute to cellulose breakdown and methane production in bovines;
culturing isolated species of bacteria; and
assessing phage resistance of isolated species of bacteria.

14. A process for digesting organic matter, comprising:
inoculating a digester containing organic matter with an inoculum containing several different genera of phage resistant bacteria;
adding a second inoculum of phage resistant bacteria to the digester to produce one or more of carbon dioxide, hydrogen, and organic acids;
adding a third inoculum of phage resistant bacteria to the digester to convert the one or more of carbon dioxide, hydrogen, and organic acids to methane;
optimizing fermentation by controlling one or more of temperature, pH, and moisture content; and
monitoring nutrients and adding nutrients as needed to optimize fermentation.
